(19) United States
(12) Patent Application Publication
(10) Pub. No.: US 2013/0115280 A1
(43) Pub. Date: May 9, 2013

(54) PHARMACEUTICAL AND/OR DIETARY COMPOSITIONS BASED ON SORT CHAIN FATTY ACIDS

(71) Applicant: Luigi Moro, Lainate (MI) (IT)

(72) Inventor: Luigi Moro, Lainate (MI) (IT)

(73) Assignee: COSMO TECHNOLOGIES LTD, Dublin 1 (IE)

(21) Appl. No.: 13/810,527

(22) PCT Filed: Jul. 13, 2011
(86) PCT No.: PCT/EP2011/061927

§ 371 Date: Jan. 16, 2013

(30) Foreign Application Priority Data
Jul. 29, 2010 (RU) 2010132133
Aug. 3, 2010 (IT) MI2010A001477

(51) Int. Cl. A61K 31/19

(52) U.S. Cl. CPC A61K 31/19
USPC 424/451; 424/464; 424/484; 514/557

(57) ABSTRACT

Pharmaceutical and/or dietary compositions based on short chain fatty acids or salts, esters and/or amides thereof in combination with one or more dietary soluble or water-dispersible fibre and at least one flavouring agent are disclosed.

Short-chain fatty acids (SCFA) are linear or branched C1-C5 monocarboxylic organic acids such as acetic, propionic, butyric, and isovaleric acids.

They are produced by the fermentation of undigested sugars and of dietary fibres within the large intestine by means of the saprophytic bacterial flora living in the colon. The production of short-chain fatty acids takes place along the entire large intestine with a gradient which decreases from the ileocaecal valve to the rectum. At the moment at which these short-chain fatty acids come into contact with the epithelial cells (colocytes) of the mucosa of the colon, they are rapidly captured within the cells where they are metabolized to acetyl-CoA, which is a fundamental factor of energetic metabolism. Of the four short-chain fatty acids mentioned above, butyric acid is considered the most important source of energy for the colocytes since it is responsible for about 70% of their oxygen consumption. About 70-90% of all of the butyric acid produced in the colon is metabolized by the colocytes (Velazquez O. C. et al, Dietary Fiber in Health and Disease, Plenum Press, N. Y., 1977, 1.23-134; Wachtershauser A. et al., Eur. J. Nutr., 2000, 39, 164-171).

Short-chain fatty acids are considered to be the main source of energy for the cells of the mucosa of the colon, and also to be fundamental factors in the control of the growth, differentiation and protection of the mucous membrane itself.

In fact a lack or substantial reduction thereof is often correlated with many functional disorders or organic pathological conditions such as, for example, disorders due to altered intestinal regularity, inflammatory intestinal conditions, ulcerative colitis, Crohn's disease, colonic neoplasias, etc. SCFAs and butyric acid or its salts in particular also intervene in the regulation of the proliferation of colonic epithelial cells, not only favouring processes of re-epithelialisation of the normal mucosa, but also inhibiting the proliferation of tumour cells, particularly by inhibition of the synthesis of the DNA of the tumour cell and by re-establishment of its natural apoptosis (Wachtershauser A. et al, Eur. J. Nutr., 2000, 39, 164-171). Bearing in mind the key role played by butyric acid in the regulation of these extremely important biological activities of the colon, its administration, in conditions of absolute or relative deficit, represents an action of fundamental importance.

The endogenous production of butyric acid requires the presence of soluble dietary fibres which are fermented for this purpose by the bacterial flora of the colon.

As well as being subject to fermentation by the bacterial flora, and thus leading to the production of endogenous butyric acid, inulin in particular, amongst the soluble dietary fibres, is itself an important factor stimulating saprophytic bacterial growth, thus helping to promote bacterial colonization and to regulate the equilibrium of the bacterial flora of the intestine (Gibson, R. G. et al., Gastroenterology, 1995, 108, 975-982; Nyman M. Br. J. Nutr. 2002, 87, s163-168).

The alimentary supply of short-chain fatty acids and of fibres can therefore be considered a constant need, even for subjects who do not show signs of disorders or pathological conditions at intestinal level, because of ever more frequent recourse to incorrect eating habits, to inappropriate dietary regimes, and to the use of ever more refined foodstuffs which are less and less rich in roughage and coarse fibre in particular. In many cases, in spite of the presence of a normal supply of fibres, the fermentation process itself may be deficient and may not lead to sufficient production of butyric acid. This reduced or absent intestinal fermentation activity is, in most cases, caused by qualitative and quantitative modifications of the bacterial flora of the intestine which are due in turn to the ingestion of substances which inhibit the development and normal growth of the flora, such as antibacterial agents, preservatives, antibiotics, etc.

As a result of this general dietary-fermentary impoverishment, the production of butyric acid may therefore be reduced to levels such as not to supply adequate energy and protection to the intestine.

The breakdown of the delicate balance of interaction between exogenous factors (dietary fibre) and endogenous factors (bacterial flora) may therefore lead to the appearance of the above-mentioned organic or functional alterations affecting the intestine and, in particular, the colon.

In the presence of a reduced or insufficient colonic endolumenal concentration of butyric acid, the most appropriate action is therefore the supply of a sufficient quantity of exogenous butyric acid, directly into the colon.

Currently, compositions based on butyric acid alone or on its Na+, Ca++, and Mg++ salts are available and, due to the extensive early absorption of the orally intaken SCFA, the only route which can ensure that appropriate concentrations of that acid reach the interior of the colon is the rectal route which, however, since it does not enable the proximal part of the colon to be reached, limits the supply purely to the distal colon, with the understandable and considerable inconvenience connected with this administration route.

Moreover, short chain fatty acids, and particularly butyric acid, are known to posses a very unpleasant smell and acrid taste, with a sweetish aftertaste (similar to ether), thanks to which they can be detected by mammal, humans in particular, also at very low concentrations (i.e. 10 ppm). Such an unfavourable characteristic leads to various difficulties in handling these compounds, particularly when used as components or active ingredients in the preparation of pharmaceutical and/or dietary compositions. In such cases, in fact, the unpleasant smell of SCFAs, butyric acid in particular, causes slackening and inaccuracy during all the phases of the manufacturing process as well as during the final phase of packaging and storage.

Bearing in mind the impracticality of this latter administration route for supplementary dietary purposes, the need to limit the energy deficit and to re-establish intestinal balance, and the difficulties which occur during the manufacturing and packaging processes, it has now surprisingly been found and constitutes a subject of the present invention that the combination of at least one short chain fatty acid such as, for example, butyric acid itself, or of a salt, an ester or amide thereof, with at least one soluble or water-dispersible dietary fibre such as, for example, inulin, and at least one flavouring agent such as, for example, vanilla essence, in an oral formulation leads to a very significant synergic effect between these components, leading to amplification of the effects that may be produced by the administration of the individual substances and to the improvement of the manufacturing and packaging processes for this kind of orally administrable products.

The combination according to the invention in fact leads to a synergy of the effects of these substances which thus make up for the energy and protective deficit due to the lack or reduced production of endogenous butyric acid and ensure an optimized final product.

A subject of the present invention is therefore oral pharmaceutical and/or dietary compositions containing at least one short-chain fatty acid, in particular butyric acid, or a salt, ester or amide thereof, in combination with at least one soluble or water-dispersible dietary fibre, in particular inulin, and at least one flavouring agent.

The oral pharmaceutical and/or dietary compositions of the invention can be formulated in form of tablet, capsule, granule or micro-granule, preferably in form of tablet.

Short chain fatty acid according to the present invention can be selected from linear or branched C1-C5 monocarboxylic organic acid, preferably from acetic acid, propionic acid, butyric acid isovaleric acid or a mixture thereof, more preferably is butyric acid.

Soluble or water-dispersible dietary fibre according to the present invention can be selected from inulin, pectin, dextrin, maltodextrin, or derivatives and mixture thereof, preferably inulin.

According to the present invention, useful flavouring agents cab be selected from natural flavours, natural essences, extractable essences, essential oils or a mixture thereof. Preferably, said at least one flavouring agent is selected from vanillin, vanilla essence, geraniol, geranium essence, eucalyptol essential oil, almond oil, fruit flavours, honey or a mixture thereof. According to the present invention, the short chain fatty acid is present in an amount ranging from 5 to 60% by weight, preferably from 10 to 50% by weight; the soluble or water-dispersible dietary fibre is present in an amount ranging from 5 to 50% by weight, preferably from 10 to 30% by weight; and the flavouring agent is present in an amount ranging from 0.01 to 3%, with respect to the total weight of the composition.

The above active components according to the invention can be used in the most appropriate physical state for the production of a suitable form for administration; since the food supplement or the pharmaceutical composition of the invention is intended for oral administration, the preferred form is the solid form.

In order to produce these solid forms, in particular the tablet form, since short chain fatty acids, particularly butyric acid, are liquids, a solid salt of the acid such as, for example, calcium butyrate, sodium butyrate, or magnesium butyrate may be used, or the acid itself may be supported on a solid substrate of inert material by the known spray-dry technique or by adsorption.

As solid substrates according to the invention, it is possible to use the excipients that are normally used for the preparation of tablets such as, for example, gum arabic, maize starch, pre-gelatinized starch, pectin, monosaccharide and polysaccharide sugars, alginates, microcrystalline cellulose, alkyl derivatives or hydroxyalkyl derivatives of cellulose with low, medium and high viscosity, monoprotic and polyprotic mineral salts, cyclodextrin, alkylcyclodextrin, hydroxyalkylcyclodextrin, pyrrolidones or derivatives, monocarboxylic organic salts and/or esters, polycarboxylic organic salts and/or esters, inorganic substrates such as colloidal silica, talc, and organic and inorganic ion-exchange resins.

In order to produce a powder from a liquid, atomization is therefore performed by the drying of a suspension of the liquid short chain fatty acid, preferably butyric acid, and solid substrate by the spray-dry technique, or the same is adsorbed on one of the above-mentioned substrates.

In both cases, a powder containing proportional quantities of the short chain fatty acid, preferably butyric acid, dispersed in the solid substrate is obtained.

In a preferred embodiment, the compositions of the invention are preferably formulated in a unitary-dose form for oral administration which can reach the specific colonic section of the intestine almost intact, or in a manner such that most of the active ingredients reach the colon cavity directly, thus passing through the gastric portion and the first portion of the intestinal tract.

This requirement takes account of the fact that, when short chain fatty acids, preferably butyric acid or its salts, are administered orally (for example in capsules or tablets) they are absorbed very rapidly and completely by the small intestine to the extent that they do not reach the colon.

This can be achieved by the use of controlled-release, delayed release, modified release, gastro-protection and/or taste-masking techniques which have their characteristic target site in the colonic section. These techniques are known in the pharmaceutical field and are normally used to formulate active substances of other types which require a specific release time and/or site such as, for example, intestinal anti-inflammatories (Brunner N. et al., Aliment. Pharmacol. Ther., 2003, 17, 395-402), systemic anti-inflammatories, anti-ulcerative agents, anti-microbial agents, or substances for energizing the mucous membrane.

European patent application EP1183014, which is incorporated herein by reference, describes, for example, a multi-matrix controlled-release technique which is known by the trade mark MMX and is characterized by the dispersion of the active ingredient in a successive and progressive mixture of three different, interconnected matrices.

Thus, according to a further embodiment, the composition of the invention comprises:
a) a matrix containing lipophilic compounds with melting point lower than 90° C., and optionally amphiphilic compounds, in which the active ingredient/s is/are at least partially inglobated;
b) optionally an amphiphilic matrix;
c) an outer hydrophilic matrix in which the lipophilic matrix and the optional amphiphilic matrix are dispersed;
d) optionally other excipients;
e) optionally a coating;

The amphiphilic compounds which can be used according to the invention comprise polar lipids of type I or II (lecithin, phosphatidylcholine, phosphatidylethanolamine), ceramides, glycol alkyl ethers such as diethylene glycol monomethyl ether (Transcutol (R)).

The lipophilic matrix consists of substances selected from unsaturated or hydrogenated alcohols or fatty acids, salts, esters or amides thereof, fatty acids mono-, di-or triglycerids, the polyethoxylated derivatives thereof, waxes, ceramides, cholesterol derivatives or mixtures thereof having melting point within the range of 40 to 90 C, preferably from 60 to 70 C.

The hydrophilic matrix consists of excipients known as hydrogels, i. e. substances which when passing from the dry state to the hydrated, one, undergo the so-called “molecular relaxation”, namely a remarkable increase in mass and weight following the coordination of a large number of water molecules by the polar groups present in the polymeric chains of the excipients themselves.

Examples of hydrogels which can be used according to the invention are compounds selected from acrylic or methacrylic acid polymers or copolymers, alkylvinyl polymers, hydroxyalkyl celluloses, carboxyalkyl celluloses, polysaccharides, dextrins, pectins, starches and derivatives, natural or synthetic gums, alginic acid.

Coating which can be used for the invention are coating able to delay, modify and/or control the release of the active ingredient/s and/or taste-mask the active ingredient unpleasant characteristics. Preferably, the coating according to the invention is a gastro-resistant coating.

Examples of gastro-resistant coating which can be used for the invention are acrylic and/or methacrylic acids polymers (Eudragit (R)) or cellulose derivatives, such as for example cellulose acetophtalate, hydroxypropyl cellulose, hydroxypropyl methyl cellulose, hydroxyethyl cellulose or a mixture thereof.

Other techniques which can be suitable for the formulation of the composition of the invention are described in EP572942 and WO 00/28974, which are also incorporated herein by reference.

According to one embodiment of the invention, the above at least one flavouring agent can be dispersed in one of the above lipophilic matrix, amphiphilic matrix, hydrophilic matrix or in all of them.

According to a further embodiment of the invention, said at least one flavouring agent can be totally, or partially, dispersed in the coating.

According to the invention, part of said at least one flavouring agent can be thus dispersed in one and/or more of the above matrices, and a part can be dispersed in the coating.

These techniques can bring about protection of the active ingredient/s throughout the transit through the stomach and during the passage through the first sections of the small intestine (the duodenum and the jejunum in particular) in order to release them directly in contact with the wall of the large intestine, precisely where their maximum concentration is required for an optimal effect.

These techniques are characterized by progressive and slow erosion of the composition, preferably a tablet or other suitable solid form for the time necessary for the gastrointestinal transit, ensuring optimal and uniform distribution of the active ingredient/s along the entire mucous membrane of the colonic section.

It has thus been possible to provide a local topical treatment, utilizing to the maximum the energizing and protective capacities of the short chain fatty acids, preferably butyric acid, which can thus act directly on the specific section of the mucous membrane of the colon, in combination with those of the soluble or water-dispersible dietary fibre, preferably inulin, and the flavouring agent which are thus brought into contact with the bacteria which can ferment it directly and produce further quantities of short-chain fatty acid.

On the basis of the foregoing, a further subject of the present invention is therefore a controlled-release, delayed-release, modified-release, taste-masking and/or gastro-resistant oral pharmaceutical and/or dietary compositions containing at least one short-chain fatty acid, at least one soluble fibre or water-dispersible dietary fibre and at least one flavouring agent, which can pass intact through the entire gastric section and the first intestinal section without disintegrating and can release the active ingredients directly at colonic level. Preferably, the composition of the invention is in tablet form.

A further object of the present invention is an oral pharmaceutical and/or dietary composition above described for use in the treatment of intestinal disorders, inflammatory bowel disorders, and pathological conditions of the intestinal mucous membrane and/or for use in prevention or treatment of intestinal neoplasias.

Preferably, the oral pharmaceutical and/or dietary composition above described is for use in the treatment of intestinal disorders, inflammatory bowel diseases or disorders, irritable bowel syndrome, actinic colitis, post-antibiotic dismicrobism and dismetabolism recovery, acute and chronic diarrhoeal disorders and pathological conditions of the intestinal mucous membrane.

A further object of the invention is a process for the preparation of the above mentioned oral pharmaceutical and/or dietary composition containing at least one short chain fatty acid, at least one soluble or water-dispersible dietary fibre, and at least one flavouring agent which comprises the following steps:

1) mixing the at least one short chain fatty acids, the at least one soluble or water-dispersible dietary fibre, the amphiphilic substance(s), the lipophilic substance(s) and optionally a part of excipients till an homogeneous mixture is obtained

2) to the previously obtained matrix, adding the hydrophilic substance(s) and optionally the other excipients to obtain the final form.

The multi-matrix compositions obtained can be then subjected to one, or more, coating step in order to obtain the controlled-release, delayed-release, modified-release, taste-masking and/or gastro-protection of the active ingredient(s) therein contained. A supplementary flavour coating can be optionally added on the surface of said composition, preferably in case of a tablet composition.

The coating of the invention can be performed using known techniques as for example, pan coat, fluid bed equipped with suitable nozzle and/or pump systems.

The following examples are included to illustrate the invention further without being limiting thereof.

### EXAMPLES

### Example 1

### Gastro-Protected, Controlled-Release Tablet

| Ingredients | Unitary amount (mg/tab.) |
|---|---|
| Calcium butyrate | 307.50 |
| (i.e. butyric acid 250 mg/tab.) | |
| Corn starch | 37.50 |
| Maltodextrins | 200.00 |
| Citric acid | 22.50 |
| Microcrystalline cellulose | 50.00 |
| Inulin | 50.00 |
| Sorbitol | 105.00 |
| Hydroxyethyl cellulose | 40.00 |
| Stearic acid | 17.00 |
| Lecithin | 5.00 |
| Colloidal Silicon dioxide | 10.00 |
| Magnesium stearate | 7.50 |
| Vanilla essence | 3.00 |
| coating composition: | |
| Shellac | 17.50 |
| Talc | 20.00 |
| Titanium dioxide | 4.00 |
| Hydroxypropylcellulose | 4.00 |
| Triethylcitrate | 4.00 |
| Vanilla essence | 4.00 |
| Ethylic alcohol | q.s. |

1.000 tablets were prepared and coated with an unitary dosage of 250 mg/cpr in butyric acid and a small amount of Inulin and Vanilla essence. The tablets are thus packaged in blister of Aluminium/PVC/PE.

The addition of vanilla essence in the matrix mixture and in the coating suspension allows to minimize the unfavourable smell of butyric acid, and to avoid olfaction problems during the final phase of the manufacturing process and packaging. The stability of the product during the storage at different conditions results to be very good, within the 10% limit usually used for the stability evaluation in pharmacological and medical fields.

To obtain the coated tablets, the following process has been applied:

Calcium Butyrate, corn starch, maltodextrins, inulin stearic acid, lecithin, citric acid and sorbitol are wet granulated using a suspension of low viscosity hydroxyethyl cellulose, After drying, the composition is completed with the addition of microcrystalline cellulose, colloidal silicon dioxide and magnesium stearate. After blending, the composition is subjected to be tabletted. The mixtures obtained are then coated with a alcoholic suspension of shellac, containing also hydroxypropylcellulose, titanium dioxide, talc, triethylcitrate and vanilla essence

The obtained tablets show a prolonged release dissolution profile, with less than 40% release in 2 hours, using disintegration test as evaluation apparatus and buffer pH 6,8 as medium.

After coating, the tablets are packaged in blister and subjected to stability evaluation.

### Example 2

### Gastro-Protected Controlled-Release Tablet

| Ingredients: | Unitary amount (mg/tab.) |
|---|---|
| Calcium butyrate | 307.50 |
| (i.e. butyric acid 250 mg/cpr) | |
| Corn starch | 37.50 |
| Maltodextrins | 210.00 |
| Citric acid | 22.50 |
| Cellulose microcrystalline | 50.60 |
| Inulin | 250.00 |
| Sorbitol | 146.25 |
| Idroxyethyl cellulose | 60.00 |
| Stearic acid | 7.50 |
| Lecithin | 5.00 |
| Colloid silica hydrate | 10.00 |
| Magnesium stearate | 7.50 |
| coating composition: | |
| Shellac | 17.4 |
| Talc | 21.3 |
| Titanium dioxide | 4.2 |
| Idroxypropylcellulose | 4.2 |
| Trietilcitrate | 4.2 |
| Vanillin | 3.9 |
| Ethylic alcohol | q. s. |

1000 tablets were prepared and coated with an unitary dosage of 250 mg/cpr in butyric acid and inulin and a smaller amount of vanillin. The tablets are thus packaged in blister of Aluminium/PVC/PE.

The addition of vanillin in the coating suspension allows to minimize the unfavourable smell of butyric acid, and to avoid olfaction problems during the final phase of the manufacturing process and packaging. The stability of the product during the storage at different conditions results to be very good, within the 10% limit usually used for the stability evaluation in pharmacological and medical fields.

### Example 3

### Gastro-Protected Controlled-Release Tablet

| Ingredients: | Unitary amount (mg/tab.) |
|---|---|
| Calcium butyrate | 615.00 |
| (i.e butyric acid 500 mg/cpr) | |
| Corn starch | 37.50 |
| Maltodextrins | 160.00 |
| Citric acid | 22.50 |
| Cellulose microcrystalline | 50.61 |
| Inulin | 50.00 |
| Sorbitol | 146.25 |
| Idroxyethyl cellulose | 60.00 |
| Stearic acid | 7.50 |
| Lecithin | 5.00 |
| Colloid silica hydrate | 10.00 |
| Magnesium stearate | 7.50 |
| coating composition: | |
| Shellac | 17.4 |
| Talc | 21.348 |
| Titanium dioxide | 4.185 |
| Idroxypropylcellulose | 4.185 |
| Trietilcitrate | 4.185 |
| Vanilla essence | 3.900 |
| Ethylic alcohol | 315 |

1000 tablets were prepared and coated with an unitary dosage of 500 mg/cpr in butyric acid and 50 mg/tab. of inulin and a smaller amount of vanilla essence. The tablets are thus packaged in blister of Aluminium/PVC/PE.

The addition of vanilla essence in the coating suspension allows to minimize the unfavourable smell of butyric acid, and to avoid olfaction problems during the final phase of the manufacturing process and packaging. The stability of the product during the storage at different conditions results to be very good, within the 10% limit usually used for the stability evaluation in pharmacological and medical fields.

### Example 4

### Gastro-Protected Controlled-Release Tablet

| Ingredients: | Unitary amount (mg/tab.) |
|---|---|
| Calcium butyrate | 307.50 |
| (i.e butyric acid 250 mg/cpr) | |
| Corn starch | 37.50 |
| Maltodextrins | 210.00 |
| Citric acid | 22.50 |
| Cellulose microcrystalline | 50.60 |
| Inulin | 250.00 |
| Sorbitol | 146.25 |
| Idroxyethyl cellulose | 60.00 |
| Stearic acid | 7.50 |
| Lecithin | 5.00 |
| Colloid silica hydrate | 10.00 |
| Magnesium stearate | 7.50 |
| Coating composition - step A: | |
| Shellac | 12.0 |
| Talc | 17.0 |
| Titanium dioxide | 4.5 |
| Trietilcitrate | 3.7 |
| Ethylic alcohol | q.s. |
| Coating composition - step B: | |
| Shellac | 2.0 |
| Vanillin | 4.0 |
| Hydroxypropylcellulose | 3.8 |
| Talc | 2.0 |
| Ethylic alcohol | q.s. |

10.000 tablets were prepared and coated with an unitary dosage of 250 mg/cpr in butyric acid and inulin and a different process to add the flavouring agent to the tablets. In facts, the here described composition foresees the application of the coating in 2 steps: the first step is including the compounds able to delay and prolong the active ingredient release from the tablet to the environment and the second coating composition, applied sequentially over the coated tablets, is including the flavouring agent vanillin with a small amount of hydrophilic polymers used to graft the flavouring agent itself to the tablet coating surface.

The two steps film coating application does not alter the dissolution characteristics of the tablets, that showed in both cases, with and without the step B coating application, the same prolonged release dissolution profile, with less than 40% release in 2 hours, using disintegration test as evaluation apparatus and buffer pH 6.8 as medium The tablets are thus packaged in blister of Aluminium/PVC/PE.

The addition of vanillin in the coating suspension with separate step allows to minimize the unfavourable smell of butyric acid maintain with the minimal change of the manufacturing process steps and without any minimal impact on the stability of the product. In fact the accelerated stability of the product during the storage at different conditions results to be very good, surely within the 10% limit usually used for the stability evaluation in pharmacological and medical fields.

### Example 5

### Gastro-Protected Controlled-Release Tablet

| | Unitary amount |
|---|---|
| Ingredients: | (mg/tab) |
| Calcium butyrate | 307,517 |
| i.e. Butyric acid 250 mg/tab | |
| Inulin | 250,000 |
| Corn starch | 50,000 |
| Maltodextrin | 300,000 |
| Citric acid | 30,000 |
| Microcrystalline cellulose | 67,483 |
| Sorbitol | 195,000 |
| Hydroxypropyl Methyl cellulose | 80,000 |
| Stearic acid | 5,000 |
| Colloidal silicon dioxide | 20,000 |
| Lecithin | 5,000 |
| Magnesium stearate | 10,000 |
| Vanilla essence | 4,000 |
| Coating composition step A: | |
| Shellac | 14,000 |
| Talc | 17,000 |
| Titanium dioxide | 4,500 |
| Hydroxypropyl cellulose | 1,800 |
| Triethylcitrate | 3,700 |
| Ethylic alcohol | q.s. |
| Coating composition step B: | |
| Talc | 17,000 |
| Hydroxypropyl cellulose | 1,800 |
| Shellac | 2,000 |
| Stearic acid | 2,000 |
| Honey aroma | 4,000 |
| Ethylic alcohol | q.s. |

10.000 tablets were prepared and coated applying the same coating process described in Example. 4. In facts, the coating application has been carried out in 2 steps: the first step is including the compounds able to delay and prolong the active ingredient release from the tablet to the environment and the second coating composition, applied sequentially over the coated tablets, is including the flavouring agent with a small amount of hydrophilic polymers used to graft the flavouring agent itself to the tablet coating surface.

The two steps film coating application does not alter the dissolution characteristics of the tablets, that showed in both cases, with and without the step B coating application, the same prolonged release dissolution profile, with less than 40% release in 2 hours, using disintegration test as evaluation apparatus and buffer pH 6.8 as medium The tablets are thus packaged in blister of Aluminium/PVC/PE to obtain the better stability profile.

1-16. (canceled)

17. Oral pharmaceutical and/or dietary composition containing butyric acid or salts thereof in combination with at least one soluble or water-dispersible dietary fibre selected among inulin, maltodextrin or a mixture thereof, at least one flavouring agent selected among vanillin, vanilla essence or a mixture thereof and one or more pharmacologically acceptable excipients, characterised in that it comprises:
a) a matrix consisting of lipophilic compounds with melting point lower than 90 C and an amphiphilic matrix, wherein the active ingredient is at least partially inglobated;
b) an amphiphilic matrix;
c) an outer hydrophilic matrix wherein the lipophilic matrix and the amphiphilic matrix are dispersed;
d) optionally other excipients;
e) a coating,
and part of said at least one flavouring agent is dispersed in one or more of said matrices, and part is dispersed in said coating.

18. Composition according to claim 17, wherein said butyric acid salts are selected among calcium butyrate, sodium butyrate or magnesium butyrate.

19. Composition according to claim 17, wherein a quantity of from 5 to 60% by weight of the butyric acid or salts thereof is included, preferably from 10 to 50% by weight, with respect to the total weight of the composition.

20. Composition according to claim 17, wherein a quantity of from 5 to 50% by weight of the soluble or water-dispersible dietary fibre is included, preferably from 10 to 30% by weight, with respect to the total weight of the composition.

21. Composition according to claim 17, wherein a quantity of from 0.001% to 5% by weight, of the flavouring agent is included, preferably from 0.01 to 3% by weight, with respect to the total weight of the composition.

22. Composition according to claim 17 in tablet, capsule, granule or micro-granule form.

23. Composition according to claim 17 wherein said coating is selected among a controlled-release coating, a delayed release coating, a modified release coating, a taste-masking coating and/or a gastro-resistant coating.

24. Composition according to claim 17, wherein it is a controlled-release, delayed-release, modified-release, taste-masking and/or gastro-resistant composition.

25. A method of treating intestinal disorders, inflammatory bowel diseases or disorders, irritable bowel syndrome, actinic colitis, post-antibiotic dismicrobism and dismetabolism recovery, acute and chronic diarrhoeal disorders and pathological conditions of the intestinal mucous membrane which comprises administering a therapeutically effective amount of the oral pharmaceutical and/or dietary composition according to claim 17.

26. A method of preventing or treating intestinal neoplasias which comprises administering an effective amount of the oral pharmaceutical and/or dietary composition according to claim 17.
